(19) United States
(12) Patent Application Publication
(10) Pub. No.: US 2026/0335677 A1
(43) Pub. Date: Oct. 29, 2026

(54) SYSTEM AND METHOD FOR CONTROL OF IONIZING RADIATION DOSE IN MEDICAL APPLICATIONS USING SYNTHETIC LOCALIZERS

(71) Applicant: The General Hospital Corporation, Boston, MA (US)

(72) Inventors: Kai Yang, Winchester, MA (US); Amita Sharma, Dover, MA (US); Rajiv Gupta, Wayland, MA (US); Theodore Marschall, Medford, MA (US); Cristy Savage, Weymouth, MA (US); Karen Flynn, South Walpole, MA (US); Travis Redel, Weymouth, MA (US); Frederick McNulty, Chelsea, MA (US)

(21) Appl. No.: 19/120,191

(22) PCT Filed: Oct. 12, 2023
(86) PCT No.: PCT/US2023/076749

§ 371 Date: Apr. 10, 2025

Related U.S. Application Data
(60) Provisional application No. 63/415,568, filed on Oct. 12, 2022.

(51) Int. Cl. A61N 5/10; A61B 6/03

(52) U.S. Cl. CPC A61N 5/1065; A61B 6/032; A61N 5/103; A61N 2005/1061

(57) ABSTRACT

Methods, system, and non-transitory media are provided for tube-current-modulation (TCM) calculation in computed tomography (CT) imaging. The radiation dose exposure is adjusted by identifying dose varying anatomical markers such as arms or removable objects that are not consistently positioned between localizer and actual scanning. Provided herein are means of reducing the effect of those anatomical markers on the TCM calculation by generating a synthetic localizer from an acquired localizer of a patient wherein the anatomical marker has been modified to match the actual scanning status. Modification of the dose-varying anatomical marker may include changing the position from an arms-down to arms up position or complete image signal removal of the arms or other markers from the localizer.

### CROSS REFERENCE TO RELATED APPLICATIONS

The present application is based on, claims priority to, and incorporates herein by reference in its entirety for all purposes, US Provisional Application Serial No. 63/415568, filed Oct. 12, 2022.

### STATEMENT OF GOVERNMENT SUPPORT

N/A

### BACKGROUND

The present disclosure relates to systems and methods for controlling the dose of ionizing radiation received by a patient in a medical process using x-ray or similar sources of ionizing radiation. More particularly, the present disclosure provides systems and methods for creating and using dynamic localizers for medical applications, such as computed tomography (CT) imaging.

In CT imaging, automatic matching of the tube current to the patient attenuation is performed to balance optimal image quality and the X-ray dosage. In order to calculate the correct tube current, the patient attenuation must be known. Tube-current-modulation (TCM) is a key dose saving mechanism for CT scans. Conventionally, TCM is performed either on the basis of the expertise of the CT operator, or by evaluation of two orthogonal overview localizers. However, when multiple body parts are scanned, they are currently planned from the same localizer(s). Currently, CT systems do not allow different position localizers to plan the complete scan (e.g., two different arm positions). Thus, if an “arms out” of the scan region localizer for each body region was used, two different CT studies would be required.

To estimate tube current along the longitudinal direction, TCMs rely on patient size estimation from a pre-scan localizer radiograph. The localizer should exclude the arms for many CT scans, including the head, chest, abdomen, and spine. However, moving the arms is not always possible in situations, such as trauma, and can, in practice, be included in many localizers and/or scans. This subjects the patient to higher doses of ionizing radiation. Furthermore, when there is a mismatch in arm positioning between acquiring the localizer and performing the actual scan, the calculated dose of ionizing radiation is higher than necessary if the arms are moved out for the scan. This is a potential problem when scanning contiguous body regions, irrespective of whether the patient is able to readily move arm positions between scans. That is, either two sets of localizers must be acquired which may interfere with workflow when contrast is used, or one set of one or two localizers is acquired and the dose delivered to image the portion of the body proximate the arms in the localizer receives too high of a radiation dose when the arms are then moved for the actual imaging scan.

Similar problems exists when other hardware is moved in and out of the field of view after a localizer has been acquired. Lowering or raising the patient table would also affect the localizer, and any table movements after the localizer has been acquired will affect the dose delivered to the patient.

Thus, there is a need for improved systems and methods of configuring and coordinating TCM for CT imaging.

### SUMMARY

The present disclosure provides systems and methods for selecting and controlling radiation dose for medical procedures using ionizing radiation, such as computed tomography imaging, radiation therapy or the like, using dynamic or synthetic localizers. In one non-limiting example, a system and method are provided to detect dose-varying anatomical markers in a localizer and generate a synthetic localizer that could, should, or will be used to accommodate one or more different positions of the dose-varying anatomical markers when performing the medical procedures. In some non-limiting examples, a TCM calculation may be performed based on an adjusted position of the arms or other anatomy, which may include creating an adjusted or synthetic localizer for use in calculating the TCM.

In aspect of the present disclosure, a method is presented. The method comprises receiving a localizer of a patient in a first position and identifying one or more dose-varying anatomical markers in the localizer. The method further comprises determining a modification of the one or more dose-varying anatomical markers to change a tube-current-modulation (TCM) calculation to reduce a total dose of radiation received by the patient during a medical procedure, generating a synthetic localizer based on the modification; and generating a report including a radiation dose plan for the patient using the synthetic localizer.

In another aspect of the present disclosure, a system for modulating tube current in a computed tomography (CT) scanner is presented. The system comprises a processor configured to receive a localizer of a patient in a first position and identify one or more dose-varying anatomical markers in the localizer. The processor is further configured to determine a modification of the one or more dose-varying anatomical markers to change a tube-current-modulation (TCM) calculation to reduce a total dose of radiation received by the patient during a medical procedure, generate a synthetic localizer based on the modification, and output the synthetic localizer to calculate a radiation dose plan for the patient based on the synthetic localizer.

In another aspect of the present disclosure, a non-transitory computer readable storage medium is described. The medium has instructions stored thereon, that when executed by a computer processor, causes the computer processor to carry out steps comprising receiving a localizer of a patient in a first position and identifying one or more dose-varying anatomical markers in the localizer. The instructions further cause the processor to carry put steps comprising determining a modification of the one or more dose-varying anatomical markers to change a tube-current-modulation (TCM) calculation to reduce a total dose of radiation received by the patient during a medical procedure, generating a synthetic localizer based on the modification; and generating a report including a radiation dose plan for the patient using the synthetic localizer.

These aspects are nonlimiting. Other aspects and features of the systems and methods described herein will be provided below.

### BRIEF DESCRIPTION OF THE DRAWINGS

The foregoing features of embodiments will be more readily understood by reference to the following detailed description, taken with reference to the accompanying drawings, in which:

FIG. 1A is schematic of an example CT system.

FIG. 1B is a schematic of an example CT system including control systems and an operator workstation.

FIG. 2 is a flowchart of a method for TCM according to aspects of the present disclosure.

FIG. 3 is an example flow chart for TCM based on the presence or absence of an arm(s) in the localizer, according to aspects of the present disclosure.

FIG. 4 is an example workflow of the generative adversarial network (GAN), according to aspects of the present disclosure.

FIG. 5A is a set of localizers of a patient for scanning the head comparing the CT dose index volume (CTDIvol) between an arms-up and arms-down configuration.

FIG. 5B is a set of localizers of a patient for scanning the thoracic and lumbar spine comparing the CTDIvol between and arms down and arms up configuration.

### DETAILED DESCRIPTION

Described herein are methods, system, and non-transitory CRM for creating synthetic localizers, for example, for use with dose-varying anatomical markers present during a CT scan. Common dose-varying anatomical markers include the arms, whose presence in a localizer scan may affect TCM calculations and cause higher radiation doses than necessary. The methods, systems and non-transitory CRM proposed provides ways of mitigating the effects of these dose-varying anatomical markers by synthesizing localizers with a modification to the dose-varying anatomical markers. In a non-limiting example, these methods, systems, and non-transitory CRM may be performed with or be integrated with existing CT systems or other systems that utilize ionizing radiation.

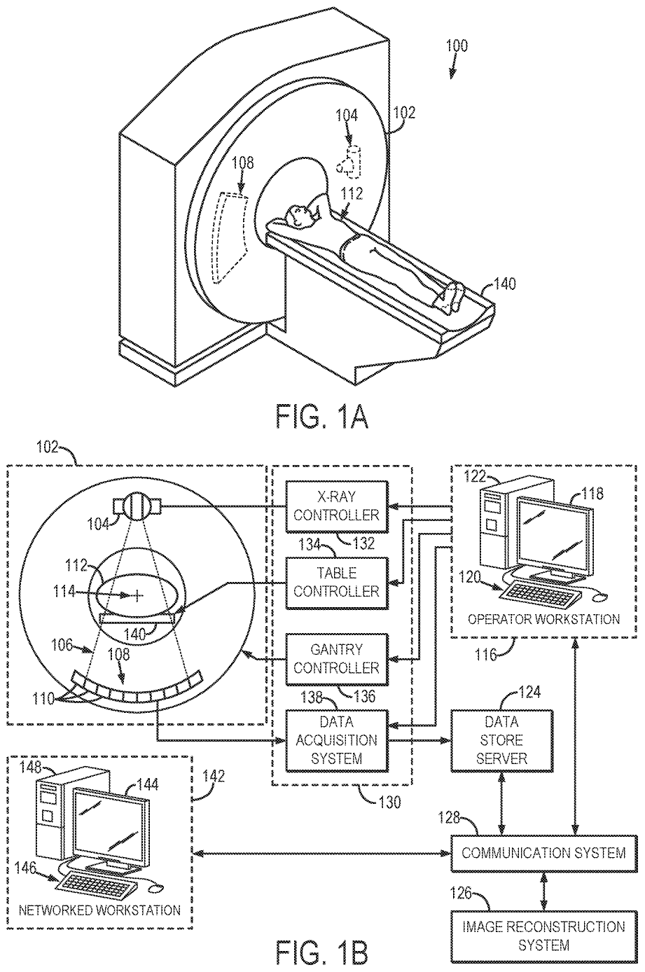

Referring to FIGS. 1A and 1B, an example of an x-ray computed tomography (“CT”) imaging system 100 is illustrated. The CT system includes a gantry 102, to which at least one x-ray source 104 is coupled. The x-ray source 104 projects an x-ray beam 106, which may be a fan-beam or cone-beam of x-rays, towards a detector array 108 on the opposite side of the gantry 102. The detector array 108 includes a number of x-ray detector elements 110. In some configurations the detector array 108 can be a multi-detector array, such that the CT system 100 is a multi-detector CT (MDCT) system. Together, the x-ray detector elements 110 sense the projected x-rays 106 that pass through a subject 112, such as a medical patient or an object undergoing examination, that is positioned in the CT system 100. In FIG. 1A the patient 112 is shown with their arms raised. In an alternative embodiment, the arms may be placed at the patient's side or crossed above the torso or chest. Each x-ray detector element 110 produces an electrical signal that may represent the intensity of an impinging x-ray beam and, hence, the attenuation of the beam as it passes through the subject 112. In some configurations, each x-ray detector 110 is capable of counting the number of x-ray photons that impinge upon the detector 110. Each x-ray detector element 110 may be an energy integrating detector (EID) or a photon counting detector (PCD). During a scan to acquire x-ray projection data, the gantry 102 and the components mounted thereon rotate about a center of rotation 114 located within the CT system 100.

The CT system 100 also includes an operator workstation 116, which typically includes a display 118; one or more input devices 120, such as a keyboard and mouse; and a computer processor 122. The computer processor 122 may include a commercially available programmable machine running a commercially available operating system. The operator workstation 116 provides the operator interface that enables scanning control parameters to be entered into the CT system 100. In general, the operator workstation 116 is in communication with a data store server 124 and an image reconstruction system 126. By way of example, the operator workstation 116, data store server 124, and image reconstruction system 126 may be connected via a communication system 128, which may include any suitable network connection, whether wired, wireless, or a combination of both. As an example, the communication system 128 may include both proprietary or dedicated networks, as well as open networks, such as the internet.

The operator workstation 116 is also in communication with a control system 130 that controls operation of the CT system 100. The control system 130 generally includes an x-ray controller 132, a table controller 134, a gantry controller 136, and a data acquisition system 138. The x-ray controller 132 provides power and timing signals to the x-ray source 104 and the gantry controller 136 controls the rotational speed and position of the gantry 102. The table controller 134 controls a table 140 to position the subject 112 in the gantry 102 of the CT system 100.

The data acquisition system (DAS) 138 samples data from the detector elements 110 and converts the data to digital signals for subsequent processing. For instance, digitized x-ray data is communicated from the DAS 138 to the data store server 124. The image reconstruction system 126 then retrieves the x-ray data from the data store server 124 and reconstructs an image therefrom. The image reconstruction system 126 may include a commercially available computer processor, or may be a highly parallel computer architecture, such as a system that includes multiple-core processors and massively parallel, high-density computing devices. Optionally, image reconstruction can also be performed on the processor 122 in the operator workstation 116. Reconstructed images can then be communicated back to the data store server 124 for storage or to the operator workstation 116 to be displayed to the operator or clinician.

The CT system 100 may also include one or more networked workstations 142. By way of example, a networked workstation 142 may include a display 144; one or more input devices 146, such as a keyboard and mouse; and a processor 148. The networked workstation 142 may be located within the same facility as the operator workstation 116, or in a different facility, such as a different healthcare institution or clinic.

The networked workstation 142, whether within the same facility or in a different facility as the operator workstation 116, may gain remote access to the data store server 124 and/or the image reconstruction system 126 via the communication system 128. Accordingly, multiple networked workstations 142 may have access to the data store server 124 and/or image reconstruction system 126. In this manner, x-ray data, reconstructed images, or other data may be exchanged between the data store server 124, the image reconstruction system 126, and the networked workstations 142, such that the data or images may be remotely processed by a networked workstation 142. This data may be exchanged in any suitable format, such as in accordance with the transmission control protocol (“TCP”), the internet protocol (“IP”), or other known or suitable protocols.

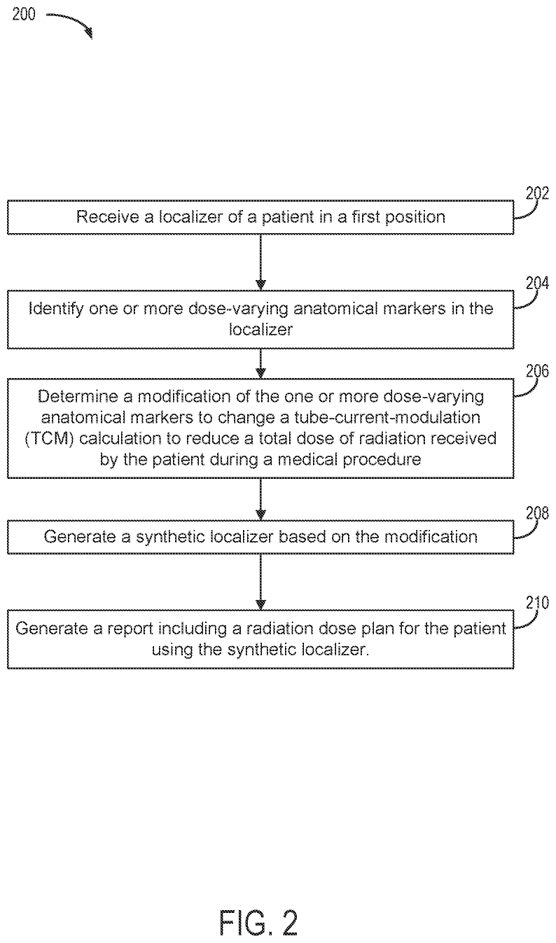

FIG. 2 is a non-limiting example of a method 200 for TCM. The method 200 includes a step 202 of receiving a localizer of a patient in a first position. A localizer scan is a two-dimensional (2D) projection image acquired using a CT scanner before the full rotational CT acquisitions. A localizer may also be referred to as a topogram, scanogram, scout, or a surview, based on the different commercial CT systems used in the field.

In a non-limiting example, the first position of a patient may be an anterior-posterior (AP) position, posterior-anterior (PA) position, or a lateral position. Furthermore, in either AP, PA, or lateral positions, the patient may have their arms raised, by their side, crossed across the chest, or hands clasped on the torso. In an alternative embodiment wherein a CT system acquires two orthogonal localizers, one of the first or second localizers may be used.

After receiving the localizer in step 202, a processor such as processor 122 of FIG. 1B may identify one or more dose-varying anatomical markers in the localizer. In a non-limiting example, a dose-varying anatomical marker may include a limb such as an arm. In another example, a dose-varying anatomical marker includes external or removable objects from the patient. Non-limiting examples include, but are not limited to, removable metal objects on the patient such as eyeglasses, jewelry, clothing, dental or orthodontic devices, and removable prosthetic devices and medical devices (e.g., insulin pumps). In one non-limiting aspect, an operator may view the localizer and manually identify the marker. Alternatively, the processor automatically identifies the marker in the localizer, for example using trained deep learning models. In one example, a convolutional neural network (CNN) based classifier is trained to identify markers in localizers. Specifically, a CNN-based classifier may be applied to the scanned region and classifies the localizer based on the presence or absence of arms or other anatomical markers.

At step 206, the processor may automatically, or a user may manually determine a modification of the one or more dose-varying anatomical markers to change a TCM calculation to reduce a total dose radiation received by the patient during a medical procedure. In a non-limiting example, the modification includes determining a second position for the patient that would change a position of the dose-varying markers to change the TCM calculation. In one aspect, this involves changing the position of the arm(s) from beside the body to above the head or vice versa. In an alternative example, the marker may be removed from the localizer by subtracting the marker image signal from the localizer to change the TCM calculation. In a further non-limiting example, the modification is based on a desired scanning program. For example, the scanning program may include, but is not limited to, CT scanning of the chest, abdomen, spine, neck, head, or whole-body. In another non-limiting example, the modification further includes determining a change in a position of the table 140 supporting the patient 112 between the localizer and the desired scanning program.

At step 208, a synthetic localizer may be generated based on the modification determined at step 206. In a non-limiting example, non-marker anatomy is preserved between the localizer and synthetic localizer, while the marker, such as a patient's arms are translated to a different position as described in step 206. Alternatively, the marker may be entirely removed from the synthetic localizer.

At step 210, the processor may generate a report indicating a radiation dose plan for the patient using the synthetic localizer. Specifically, the synthetic localizer is used to determine a scan range which is used to calculate the radiation dose. In non-limiting example, the calculated radiation dose is lower in the synthetic localizer due to the repositioning of the marker (e.g., arms) or removal of the marker signal (e.g., metal objects on patient's body during scan). Further, the processor causes the CT system to perform a CT scan based on the report, including the TCM calculation.

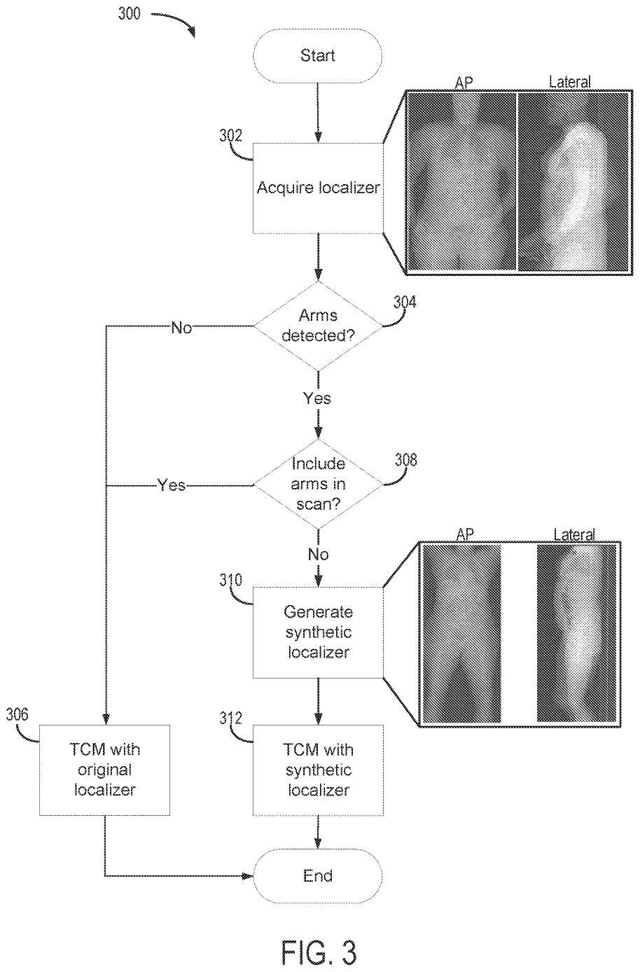

Referring now to FIG. 3, a non-limiting example workflow of TCM based on a patient's arm position is shown. At step 302, a localizer is acquired. As shown, the localizer may be an AP or lateral view with the arms down by the side of the of torso. At decision block 304, the processor analyzes the localizer for the presence of one or both arms. If arms are not detected in the localizer the TCM calculation may be based on the acquire localizer at step 306. On the other hand, if arms are detected in the localizer the processor may proceed to decision block 308 prompting a user whether to include the arms in the scan. If the arms are to be included (i.e., Yes), the TCM calculation may again be based on the acquired localizer 306.

In a non-limiting example, if the arms are not to be included in the scan at decision block 308, then the processor generates a synthetic localizer 310. As shown in FIG. 3, the synthetic localizer in either an AP or lateral view is modified such that the arms are raised above the head.

Next, at step 312, the processor may calculate the TCM with the synthetic localizer generated at step 310. Specifically, the synthetic localizer is used to determine a scan range which is used to calculate the TCM.

In a non-limiting example, the processor, such as processor 112 in FIG. 1 includes a generative adversarial network (GAN) for generating the synthetic localizer from the acquired localizer. GANs are a class of machine learning framework wherein two networks, a generator network and a discriminator network compete in the form of a zero-sum game. GANs may generate new data with the same statistics as an input training set. As used herein, the GAN is trained on localizers where the anatomical markers are not within the image plane and can generate synthetic localizers that look superficially authentic to an operator. A discriminator indirectly trains the GAN based on how realistic the synthetic localizers appear, which may be continuously updated. In a non-limiting example, the GAN is trained via unsupervised learning. Alternatively, the GAN may be trained using semi-supervised learning, fully supervised learning, and reinforcement learning.

In a non-limiting aspect, the generator network is trained by inputting one or more localizers in a first domain and creating one or more synthetic localizers in a second domain. In a non-limiting example, the generator network and the discriminator network are trained in an adversarial manner to classify the second domain localizer inputs as either real or synthetic.

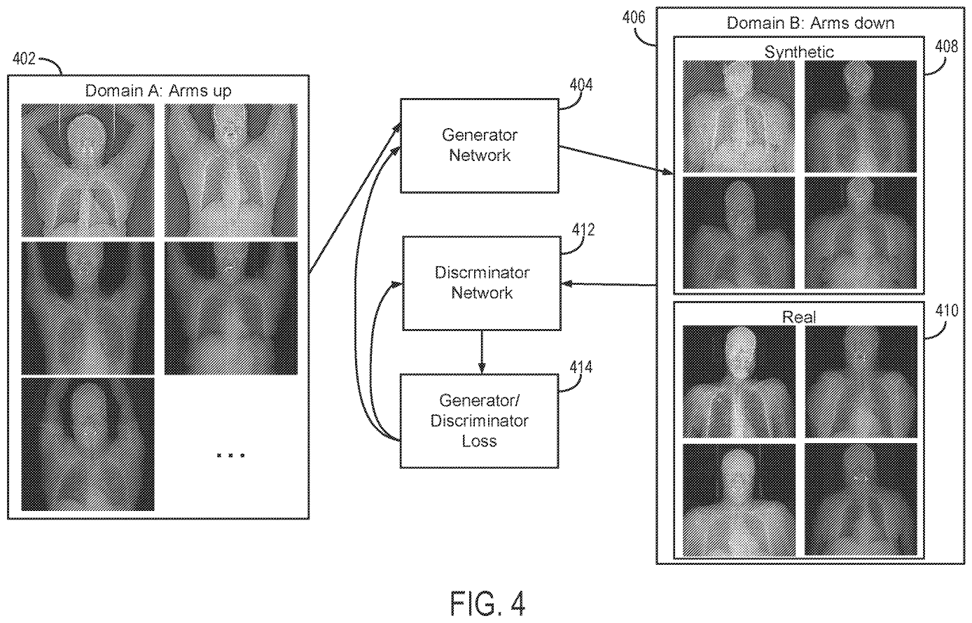

FIG. 4 shows another non-limiting example, in this case, of using the GAN to generate synthetic localizers in a head scan set-up. For a head scan, an “arms down” position is preferred to reduce the radiation dose to the target region in the neck or head. In this example, the GAN is used to synthesize one or more localizers with an “arms down” position from acquired localizers in an “arms up” position. In this example, domain A 402 includes one or more localizers of chest scans with “arms up” positioning. The localizers of domain A are input into a generator network 404 to generate one or more synthetic localizers 408 in a second domain B 406 with “arms down” positioning. Further, one or more real localizers 410 with “arms down” positioning are input into domain B 406. It is noted that the real localizers in domain A 402 and domain B 410 are unpaired, meaning they were acquired from different patients during different exams. The discriminator network 412 of the GAN then receives the synthetic localizers 408 and real localizers 410 from domain B 406 which is trained in an adversarial manner in the generator/discriminator loss block 414. The results of the generator/discriminator loss 414 are backpropagated to the generator network 404 to update the network for the next iteration of training, where a new batch of synthetic localizers are generated and input to the discriminator network 412 for further training. In short, FIG. 4 illustrates an example of an unsupervised workflow of generating synthetic localizers 408 that iteratively become less distinguishable from real localizers 410.

In a non-limiting example, the workflow of FIG. 4 may also be implemented for arm repositioning of a head scan, a thoracic and lumbar (T/L) spine scan, chest scan, or abdominal scan of an AP, PA, or lateral view of the patient, or a combined AP/PA and lateral view. Alternatively, the workflow may be used to generate synthetic localizers for TCM with the signal from the dose-varying anatomical markers removed from the original localizer.

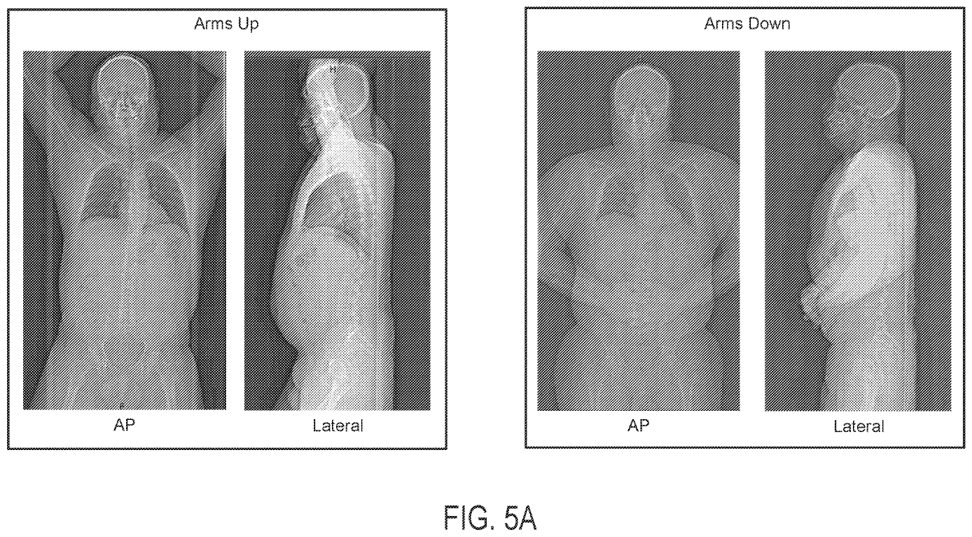

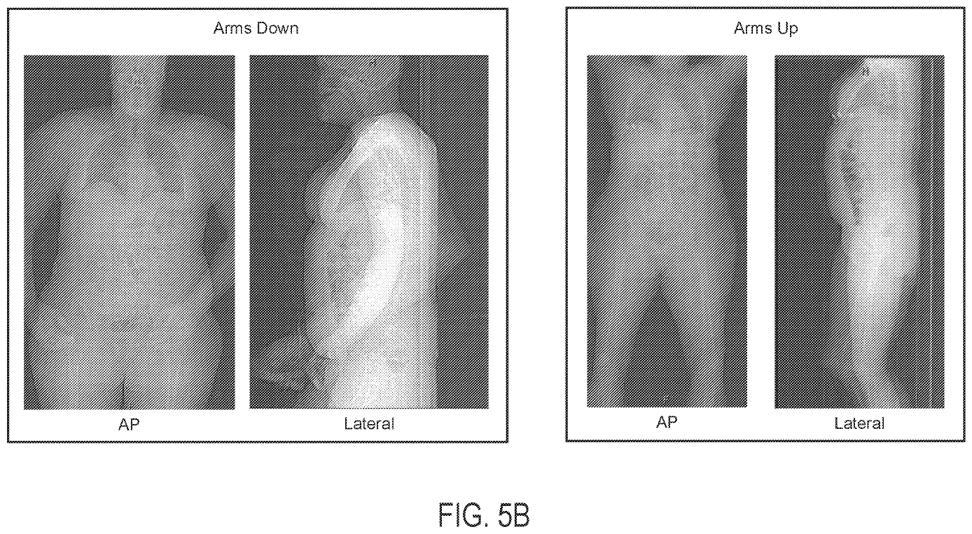

FIGS. 5A and 5B illustrate the difference in the radiation dose calculated from localizer images based on the arm position. In FIG. 5A, head scans were performed on a patient, whereby localizers of different arm positions (left panel: arms up; right panel: arms down) calculated TCM with a two to three-fold difference between them. Specifically, the CTDIvol for the head scan localizer with arms up was 140 mGy compared to the CTDIvol of 45 mGy from a head scan localizer with arms down. It is noted that the patient's arms were down during both head scan series.

Likewise, FIG. 5B shows T/L spine scan localizers of a different patient with different arm positions (left panel: arms down; right panel: arms up). The calculated CTDIvol from the localizer with arms down was 47 mGy, compared to a CTDIvol of 23 mGy calculated from the localizer with the arms up. It is further noted that the patient's arms were up during both T/L spine scan series.

Thus, systems, methods, and computer-readable medium are provided for selecting and controlling radiation dose for medical procedures using ionizing radiation, such as computed tomography imaging, radiation therapy or the like, using dynamic or synthetic localizers. In one non-limiting example, a system and method are provided to detect dose-varying anatomical markers in a localizer and generate a synthetic localizer that could, should, or will be used to accommodate one or more different positions of the dose-varying anatomical markers when performing the medical procedures. In some non-limiting examples, a TCM calculation may be calculated based on an adjusted position of the arms or other anatomy, which may include creating an adjusted or synthetic localizer for use in calculating the TCM.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (e.g., hard disks, floppy disks), optical media (e.g., compact discs, digital video discs, Blu-ray discs), semiconductor media (e.g., random access memory (“RAM”), flash memory, electrically programmable read only memory (“EPROM”), electrically erasable programmable read only memory (“EEPROM”)), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

As used in this specification and the claims, the singular forms “a,” “an,” and “the” include plural forms unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or “B” or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use an aspect of a described embodiment or feature contained in the same, or a definitive decision to use a specific skill regarding a described embodiment or feature contained in the same. In this latter context, the modal verb “may” has the same meaning and connotation as the auxiliary verb “can.”

1. A method comprising:
receiving a localizer of a patient in a first position;
identifying one or more dose-varying anatomical markers in the localizer;
determining a modification of the one or more dose-varying anatomical markers to change a tube-current-modulation (TCM) calculation to reduce a total dose of radiation received by the patient during a medical procedure;
generating a synthetic localizer based on the modification; and
generating a report including a radiation dose plan for the patient using the synthetic localizer.

2. The method of claim 1, wherein the one or more dose-varying anatomical markers includes one or more arms, removable metal objects, removable medical devices or removable prosthetic devices.

3. The method of claim 1, wherein the modification includes determining a second position for the patient that would change a position of the dose-varying anatomical markers to change the TCM calculation.

4. The method of claim 1, wherein the modification includes removing the dose-varying anatomical markers from the localizer to change the TCM calculation.

5. The method of claim 4, wherein removing the dose-varying anatomical markers from the localizer includes subtracting an image signal of the markers from the localizer.

6. The method of any one of claims 3 or 4, wherein the modification is based on a desired scanning program.

7. The method of claim 6, wherein the scanning program includes CT scanning of a chest, abdomen, spine, neck, head, or whole-body of the patient.

8. The method of claim 6, wherein the modification further includes determining a change in a position of a table supporting the patient between the localizer and the desired scanning program.

9. The method of claim 1, further comprising notifying a user of the dose-varying anatomical markers for manually determining the modification.

10. The method of claim 9, further comprising performing a CT scan based on the report.

11. A system for modulating tube current in a computed tomography (CT) scanner, the system comprising:
a processor configured to:
receive a localizer of a patient in a first position;
identify one or more dose-varying anatomical markers in the localizer;
determine a modification of the one or more dose-varying anatomical markers to change a tube-current-modulation (TCM) calculation to reduce a total dose of radiation received by the patient during a medical procedure;
generate a synthetic localizer based on the modification; and
output the synthetic localizer to calculate a radiation dose plan for the patient based on the synthetic localizer.

12. The system of claim 11, wherein the processor includes a generative adversarial network (GAN).

13. The system of claim 12, wherein the GAN includes a generator network that is trained by inputting one or more localizers in a first domain and creating one or more synthetic localizers in a second domain.

14. The system of claim 13, wherein the processor includes a discriminator network.

15. The system of claim 14, wherein the discriminator network inputs additional localizers in the second domain and the synthetic localizers from the generator network.

16. The system of claim 15, wherein the generator network and discriminator network are trained together in an adversarial manner to classify the synthetic localizers and additional localizers in the second domain as real or synthetic.

17. The system of claim 11, wherein the one or more dose-varying anatomical markers includes one or more arms, removable metal objects, removable medical devices, or removable prosthetic devices.

18. The system of claim 11, wherein the modification includes determining a second position for the patient that would change a position of the dose-varying anatomical markers to change the TCM calculation.

19. The system of claim 11, wherein the modification includes removing the dose-varying anatomical markers from the localizer to change the TCM calculation.

20. The system of claim 19, wherein removing the dose-varying anatomical markers from the localizer includes subtracting an image signal of the markers from the localizer.

21. The system of any one of claims 18 or 19, wherein the modification is based on a desired scanning program.

22. The system of claim 21, wherein the scanning program includes CT scanning of a chest, abdomen, spine, neck, head, or whole-body of the patient.

23. The system of claim 21, wherein the modification further includes determining a change in a position of a table supporting the patient between the localizer and the desired scanning program.

24. The system of claim 11, wherein the processor is further configured to notify a user of the dose-varying anatomical markers for manually determining the modification.

25. The system of claim 24, further comprising performing a CT scan based on the radiation dose plan.

26. A non-transitory computer readable storage medium having stored thereon, instructions, that when executed by a computer processor, causes the computer processor to carry out steps comprising:
receiving a localizer of a patient in a first position;
identifying one or more dose-varying anatomical markers in the localizer;
determining a modification of the one or more dose-varying anatomical markers to change a tube-current-modulation (TCM) calculation to reduce a total dose of radiation received by the patient during a medical procedure;
generating a synthetic localizer based on the modification; and
generating a report including a radiation dose plan for the patient using the synthetic localizer.

27. The medium of claim 26, wherein the one or more dose-varying anatomical markers includes one or more arms, removable metal objects, removable medical devices, or removable prosthetic devices.

28. The medium of claim 26, wherein the modification includes determining a second positions for the patient that would change a position of the dose-varying anatomical markers to change the TCM calculation.

29. The medium of claim 26, wherein the modification includes removing the dose-varying anatomical markers from the localizer to change the TCM calculation.

30. The medium of claim 29, wherein removing the dose-varying anatomical markers from the localizer includes subtracting an image signal of the marker from the localizer.

31. The medium of any one of claims 28 or 29, wherein the modification is based on a desired scanning program.

32. The medium of claim 31, wherein the scanning program includes CT scanning of a chest, abdomen, spine, neck, head, or whole-body of the patient.

33. The medium of claim 31, wherein the modification further includes determining a change in a position of a table supporting the patient between the localizer and the desired scanning program.

34. The medium of claim 26, further comprising instructions for the computer processor to notify a user of the dose-varying anatomical markers for manually determining the modification.

35. The medium of claim 34, further comprising performing a CT scan based on the report.
